(19) United States
(12) Patent Application Publication
(10) Pub. No.: US 2009/0156949 A1
(43) Pub. Date: Jun. 18, 2009

(54) Medical device with real-time physiological signal analysis function

(76) Inventors: Wei-Chih HU, Chung Li (TW); Liang-Yu Shyu, Chung Li (TW); Yong Tai Lin, Changhua City (TW); Shih Yu Lee, Dajia Township (TW)

Correspondence Address: SINORICA, LLC, 2275 Research Blvd., Suite 500, ROCKVILLE, MD, 20850, US

(21) Appl. No.: 12/102,020

(22) Filed: Apr. 14, 2008

(30) Foreign Application Priority Data
Dec. 12, 2007 (TW) 096147462

(51) Int. Cl. A61B 5/0402

(52) U.S. Cl. 600/515

(57) ABSTRACT

A medical device with real-time physiological signal analysis function is disclosed. An ECG signal is generated by detection of detection circuit to the human heart while conversion circuit receives the ECG signal and converts it into an ECG data. The ECG data is sent to a process control unit for being processed to generate a HRV parameter. The ECG data and the HRV parameter are shown by a display unit for real-time analysis of changes of HRV in time domain and frequency domain. Thus doctors can make diagnosis according to these data. Moreover, The process control unit is coupled to the memory module so as to save the ECG data and the HRV parameter. Thus after long-term data collection of the ECG and HRV, doctors can make diagnosis by means of these data.

### BACKGROUND OF THE INVENTION

The present invention relates to a medical device, especially to a medical device with real-time physiological signal analysis function.

Due to fast economics growth and quick pace of life, people always neglect their health condition. Especially after taking delicate food and without sufficient exercise for so long, people are easy to have high blood pressure, cardiovascular diseases. In recent years, there are more and more cases of “sudden death” caused by long working hours or high pressure and people are terrified.

Generally, “sudden death” is related to high pressure or long working hours. While keeping high tension under high pressure for a long period of time, people may have physical or mental health problems. The heart beating speed, physiological functions and dynamic metabolism of people are affected by emotional status, environmental factors, endocrine system, sympathetic and parasympathetic nerves. HRV (Heart Rate Variability) is the variation in times between successive heartbeats-continual change in heart rate. The value of HRV shows whether the autonomic nervous system is functioning normally and indicates conditions of the heart. When autonomic failure appears, the mechanism to maintain internal balance is broken. The decreased HRV means high risk of heart diseases and high mortality.

Through decades of research, it shows that HRV is an index of autonomic nervous activity, particularly to the parasympathetic nervous activity. It is learned that under several conditions of heart malfunction such as aging, Diabetes mellitus, heart failure, Myocardial Infarction, coronary heart disease, sudden cardiac death, chronic renal failure and obstructive lung disease, the HRV decreases. The HRV is especially useful in evaluation of prognosis of heart disease patients. It can not only work as an indicator for heart disease severity, but also in other respects such as prediction of survival rate of patients with myocardial infarction, evaluation of possibility of sudden cardiac death or ventricular fibrillation, evaluation of reinnervation or rejection in human cardiac transplant recipients.

There are several factors affected heat rate. Firstly, rhythmic discharge frequency of pacemaker cells of SA Node (Sinoatrial Node). Secondly, regulation mechanism of the Autonomic nervous system such as sympathetic nervous system that increases heart beat rate and parasympathetic nervous system that inhibits heart beat rate. The HRV is change of the discharge frequency of SA Node that is regulated by the activity of the autonomic nerve.

There are two kinds of analysis methods of HRV-one is time domain analysis while the other is frequency domain analysis. The calculation of time domain is more simple and more indicative while the sensitivity as well as the specificity is lower than other methods. Thus it's unable to distinguish action of and balance between the sympathetic nerve and the parasympathetic nerve. Therefore, the system of the present invention focuses on the frequency domain analysis. According to previous studies, there are three areas in power spectrum of the HRV: High Frequency (HF) area ranging from 0.15 to 0.4 Hz, Low Frequency (LF) area ranging from 0.04 to 0.15 Hz and very Low Frequency (VLF) area ranging from 0 to 0.04 Hz. When the variability of the HRV is quite large, the individual difference will be also obvious. Moreover, each index such as LF, HF, LF/HF, LF+HF also changes dramatically. Thus through each index of HRV, quantification of activity of sympathetic and parasympathetic nerves is easily to be achieved by the power spectrum analysis.

The HRV can be applied to analysis of diseases, physiological conditions, use of drug, heart disease prevention and prognosis evaluation. In human bodies, stable heart beat depends on a complex and interactive physiologic nervous system. The main nerve system of the heart is autonomic nerve so that the autonomic nerve plays an important role in control of heart beat rate. HRV represents changes of heart beat interval. By analysis of HRV, information related to regulation mechanism of the autonomic nervous system and its clinical effects is revealed. Moreover, whether the function of sympathetic nerve and that of parasympathetic nerve are correlated with each other can also be found out precisely so as to provide correct diagnosis and evaluation of therapeutic effects of autonomic instability.

Therefore there is a need to provide a novel medical device with real-time physiological signal analysis function that can analyze changes of HRV in time domain and frequency domain in time and collect data in the long run so that changes of HRV can be studied periodically or anytime. Thus individual difference is got. The real-time monitoring as well as analysis of HRV is also achieved.

### SUMMARY OF THE INVENTION

Therefore it is a primary object of the present invention to provide a medical device with real-time physiological signal analysis function that analyzes and shows changes of HRV on the time domain and the frequency domain in time so that doctors can make diagnosis according to these data.

It is another object of the present invention to provide a medical device with real-time physiological signal analysis function that collects ECG data and HRV parameter in the long run so that doctors can make diagnosis according to these data.

The medical device with real-time physiological signal analysis function of the present invention includes a detection circuit, a conversion circuit, a process control unit, a memory module and a display unit. The detection circuit detects heart beat of the human body to generate an electrocardiographic(ECG) signal while the conversion circuit receives the ECG signal and converts it into an ECG data. The data is sent to the process control unit for being processed to generate a HRV parameter. The display unit shows the ECG data and the HRV parameter. The process control unit is coupled to the memory module so as to save the ECG data and the HRV parameter.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein

FIG. 1 is a block diagram of an embodiment according to the present invention;

FIG. 2 is a block diagram of a detection circuit of an embodiment according to the present invention;

FIG. 3 is a block diagram of a process control unit of an embodiment according to the present invention;

FIG. 4 is a block diagram of an analysis process module of an embodiment according to the present invention;

FIG. 5 is a flow chart of an analysis process module of an embodiment according to the present invention; and

FIG. 6 is a block diagram of a peripheral control module of an embodiment according to the present invention.

### DETAILED DESCRIPTION OF THE PREFERRED EMBODIMENT

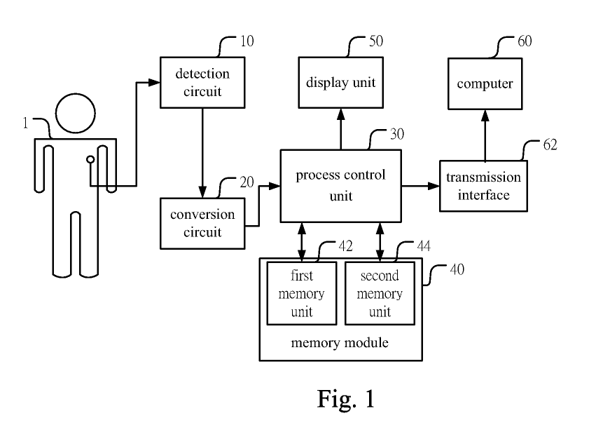

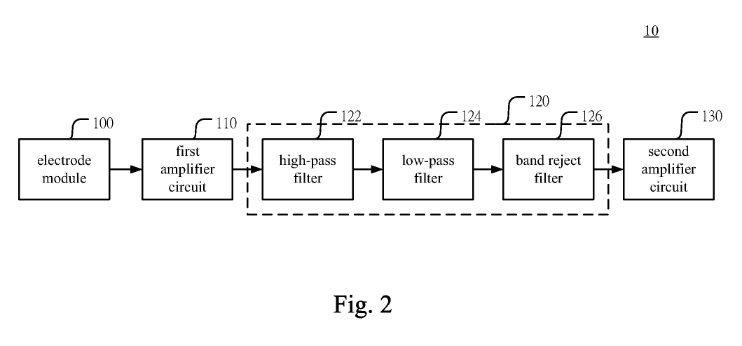

Refer to FIG. 1, a medical device with real-time physiological signal analysis function of the present invention consists of a detection circuit 10, a conversion circuit 20, a process control unit 30, a memory module 40 and a display unit 50. The detection circuit 10 detects heart beat of the human body 1 to generate an electro-cardio-graphic(ECG) signal. With reference of FIG. 2, the detection circuit 10 includes an electrode module 100, a first amplifier circuit 110, a filter circuit 120 and a second amplifier circuit 130. The electrodes of the electrode module 100 are set on tow sides of the chest and the chin is used as grounding point together with the detection circuit 10 so as to measure ECG signal of the human body 1.

The first amplifier circuit 110 is an instrumentation amplifier. Because the ECG signal is quite weak and instable, the first amplifier circuit 110 receives ECG signal detected by the electrode module 100 for amplifying weak psychological (ECG) signal while the filter circuit 120 receives the ECG signal amplified by the first amplifier circuit 110 for filtering noises of the ECG signal. The filter circuit 120 is composed of a high-pass filter 122, a low-pass filter 124 and a band reject filter 126. The high-pass filter 122 receives the amplified signal from the first amplifier circuit 110 and removes low frequency drift of the ECG signal so as to prevent interference from low-frequency. The high-pass filter 122 is a Butterworth Filter. In consideration of maintaining the ECG signal as possible and simultaneously removes unnecessary high-frequency noises, the low-pass filter 124 is added. The low-pass filter 124 receive the high-frequency part of the ECG signal filtered by the high-pass filter 122 and removes low frequency drift part of the ECG signal so as to prevent interference from high frequency mainly at 60 Hz noise caused by household electrical appliances. Most of the ECG signal falls in the frequency ranging from 1 Hz to 30 Hz so that cut-off frequency is set at 30 Hz. Thus signal at 60 Hz is filtered at once and the low-pass filter 124 works as pre-filter for filtering signal at 60 Hz. The low-pass filter 124 is a Butterworth fourth-order low-pass filter. The band reject filter 126 filters power noise at 60 Hz of the ECG signal being filtered by the high-pass filter 122. The second amplifier circuit 130 receives the ECG signal filtered by the filter circuit 120 and amplifies the filtered ECG signal.

The conversion circuit 20 receives and converts the ECG signal into an ECG data. The conversion circuit 20 is an analog to digital converter (ADC) that converts analog ECG signal into digital ECG data. Then the process control unit 30 receives and processes the ECG data to generate a HRV (Heart Rate Variability) parameter. The process control unit 30 is a System on Chip or System-on-a-chip (SoC) that is an integrated circuit for a specific target and having all components of the whole system and related software. Moreover, the process control unit 30 is a field programmable gate array (FPGA) that is a logic device configured by the end user to perform many logic functions and can be an ASIC(Application Specific Integrated Circuit) element. The FPGA is a Programmable Logic Device (PLD) based on gate array technology. FPGAs use a grid of logic gates, similar to that of an ordinary gate array, but the programming is done by the customer, outside the factory.

Therefore, by the System on Chip, change of HRV on the time domain as well as on the frequency domain is obtained.

The memory module 40 saves the ECG data and the HRV parameter. The memory module 40 includes a first memory unit 42 and a second memory unit 44, respectively being saved with the ECG data and the HRV parameter for long term data collection. When the doctor examines the patient, he/she can make diagnosis by means of the ECG data and the HRV parameter in the first memory unit 42 and the second memory unit 44. The first memory unit 42 and the second memory unit 44 can be a flash memory.

The display unit 42 is coupled to the process control unit 30 for receiving and displaying the ECG data as well as the HRV parameter. Thus while examining the patient, the doctor can make the diagnosis with reference to the display unit 42. The display unit 42 can be a Liquid Crystal Module (LCM) or a Liquid Crystal Display (LCD) 44. Furthermore, the medical device of the present invention is further coupled to a computer 60. The ECG data as well as the HRV parameter is sent to the computer 60 so as to show their change on the time domain and frequency domain. A transmission interface 62 is arranged between the medical device and the computer 60 for data transmission. The transmission interface 62 can be a universal serial bus (USB) interface, a Peripheral Component Interconnect (PCI) card, a 1394 interface, a local area network (LAN) interface (IEEE802.3), an infrared (IrDA) interface, a Bluetooth interface or others.

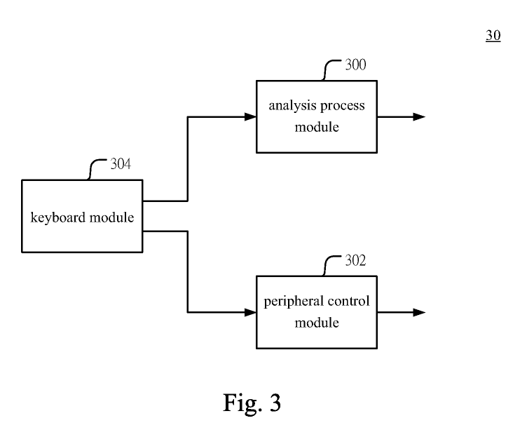

Refer to FIG. 3, the process control unit 30 consists of an analysis process module 300 and a peripheral control module 302. The analysis process module 300 receives and processes the ECG data to generate the HRV parameter while the peripheral control module 302 receives the ECG data as well as the HRV parameter and sends them to the memory module 40 and the display unit 50. The analysis process module 300 transmits data by parallel processing. Simply put, at the same time, each module runs the processing procedures according to trigger conditions of itself while in general microprocessors, processes of next module is run after processes of the previous module being finished. Thus the processing time of the SoC is shortened. Moreover, the FPGA chip runs the module at 50 MHz clock frequency so that the overall efficiency is apparently improved while the time spent is shortened dramatically so as to achieve real-time effects.

Moreover, the process control unit 30 further includes a keyboard module 304 that is coupled to the analysis process module 300 and the peripheral control module 302 for control of them respectively. The keyboard module 304 has three main functions: control of the medical device to begin the detection, control of the analysis process module 300 to start processing and analysis and control of data display. The control of the medical device to begin the detection means to control the conversion circuit 20 for converting the ECG signal. The data can be sent to the external computer 60 or shown by the display unit 50 of the medical device.

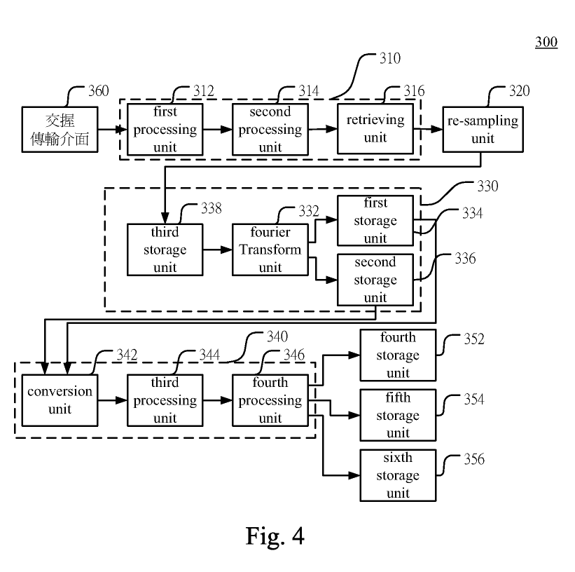

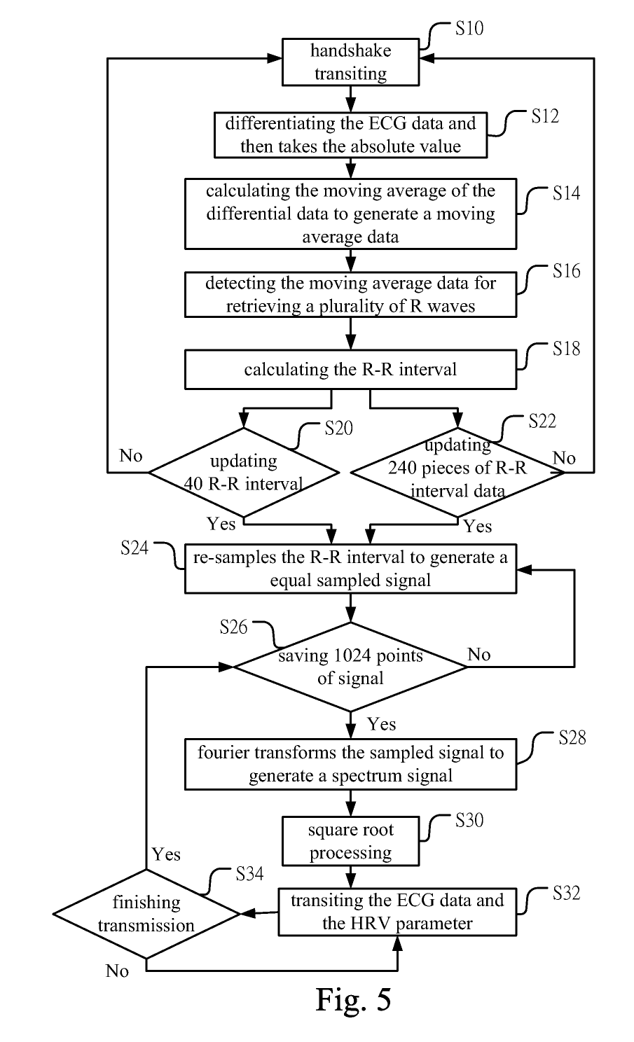

Refer to FIG. 4 & FIG. 5, the analysis process module 300 consists of a computation module 310, a re-sampling unit 320, a Fourier Transform module 330 and a square root calculation module 340. The computation module 310 receives and processes the ECG data to generate a R-R interval. That means the detection of QRS waves is performed automatically to get R wave related data for getting the R-R interval. The computation module 310 is composed of a first processing unit 312, a second processing unit 314 and a retrieving unit 316. The first processing unit 312 receives and differentiates the ECG data and then takes the absolute value to get a differential data, as shown in step S12. Because in ECG signals of some patients, the T-wave is larger than the R-wave in magnitude, or value of T-wave is close to that of R-wave, once R-wave is detected simply by setting of a threshold vale, it's easy to have errors in detection results. Thus the present invention uses differentiation as well as features of the slope to eliminate T-wave as well as P-wave interference and enhance R wave. Furthermore, for enhancing high-frequency part, take the absolute value of the differential data.

The second processing unit 314 receives and calculates the moving average of the differential data to generate a moving average data, as shown in step S14. After being processed by the second processing unit 314, the zero-crossing part is averaged and the curve becomes more smooth so that a threshold value is set for calculation of the position of R-wave. In the second processing unit 314, parallel processing is also used. The results from the first processing unit 312 are respectively saved into a register (not shown in figure). After being saved with 32 values, the register performs the moving average calculation and sends the calculation result to the next module. Afterwards, each time an absolute value of the differential data enters, the second processing unit 314 generates a result at the same time. Such parallel processing is used in each module in the analysis process module 300. Therefore, the processing time of the analysis process module 300 is reduced significantly to achieve real-time effects.

The retrieving unit 316 detects the moving average data for retrieving a plurality of R waves, as shown in step S16, to calculate the R-R interval, as shown in step S18. The retrieving unit 316 uses the previous four heart beat as reference values, being processed by the first processing unit 312 and the second processing unit 314, a maximum value is obtained. Use 50% of the maximum value as a threshold value. Once the data is over the threshold value, start counting and now the slope is positive. Time from now on until next time magnitude of the wave is over the threshold value and the slope is positive, the counting period therebetween is the detected R-R interval. Moreover, at the first time, the retrieving unit 316 needs to save 240 pieces of R-R interval data. After that, re-sampling is performed each time 40 R-R interval are updated.

The re-sampling unit 320 receives and samples the R-R interval to generate a equal sampled signal, as shown in step S24. By the window interpolation method, the re-sampling unit 320 turns the signal into the equal-time-interval sampled Heart Rate Variability parameter for convenience of power spectrum analysis.

After the re-sampling unit 320 sampling 1024 points of signal (step S26), perform the module processing. The Fourier Transform module 330 receives and transforms the sampled signal to generate a spectrum signal, as shown in step S28. The Fourier Transform module 330 consists of a Fourier Transform unit 332, a first storage unit 334 and a second storage unit 336. The Fourier Transform unit turns the sampled signal into the spectrum signal that includes a real number and an imaginary number, respectively being saved into the first storage unit 334 and the second storage unit 336. The first storage unit 334 and the second storage unit 336 are both First Input First Output (FIFO) register. Moreover, the Fourier Transform unit 332 is in a fixed-point operation and is integrating data processing procedures between the FIFO structure of the first storage unit 334 as well as the second storage unit 336 and the Fourier Transform unit 332. By the transmission principle of the FIFO, the re-sampled data is sent to a third storage unit 338 and then the Fourier Transform unit 332. After receiving 1024 pieces of data, the Fourier Transform unit 332 starts to perform parallel Fourier Transform operations. Thus 1024 results of real number and imaginary number are generated. Now in the Fourier Transform unit 332, a signal is generated to inform the Fourier Transform unit 332 of the Fourier Transform module 330 that the processing is finished In the next second, values of real number as well as imaginary number are saved into the first storage unit 334 and the second storage unit 336 in sequence. Thus the Fourier Transform unit 332 of the analysis process module 300 is quite important. The processing of each time points should be correct so that there is no error on processing results or loss of the processed value which may lead to wrong results.

Because the processing results are divided into the real numbers and imaginary numbers and the values are with sign (plus or minus) so the final results should be got by taking the square root of them. The square root calculation module 340 receives and processes the spectrum signal so as to generate the HRV parameter, as shown in step S30. The square root calculation module 340 consists of a conversion unit 342, a third processing unit 344 and a fourth processing unit 346. The design of the square root calculation module 340 is to integrate the real numbers and imaginary numbers from the Fourier Transform unit 332. By the conversion unit 342, the signed 16 bit of the fixed-point in the spectrum signal is turned into the unsigned number. Through the third processing unit 344, add the square of the unsigned number of the spectrum signal to one another to get a square sum data. Through the fourth processing unit 346, take the square root of the square sum data to generate the HRV. The final results are saved in three storage units-a fourth storage unit 352, a fifth storage unit 354 and a sixth storage unit 356. Two clocks are spent to perform the processing of the square root calculation module 340. That means it takes 40 ns (nanosecond) and the results may have a bit error. The whole system runs at 50 MHz while the square root calculation module 340 runs at 25 MHz. Thus the square root calculation module 340 plays an important role and processing at each time point should be correct. Moreover, the communication between it and the FIFO storage unit must be done carefully. Therefore, the value of the result will not have error. Later, the ECG data and the HRV parameter are sent to the computer 60 or the display unit 50 for displaying, as shown in step S32. Refer to step S34, after finishing transmission of the 1024 pieces of data, repeat the step S26.

Due to difference between the clock circuit(not shown in figure) of the process control unit 30 and that of the memory module 40, the transmission between them is asynchronous data transmission. There is no clock as the reference lock s so that a data exchange between the two ends is confirmed through a signal from a sending end and a corresponding signal from a receiving end and this way is like shaking hands. Thus this is called Handshake. Therefore, the analysis process module 300 further includes a Handshake interface that receives and sends ECG data to the memory module 40 by using a handshake procedure.

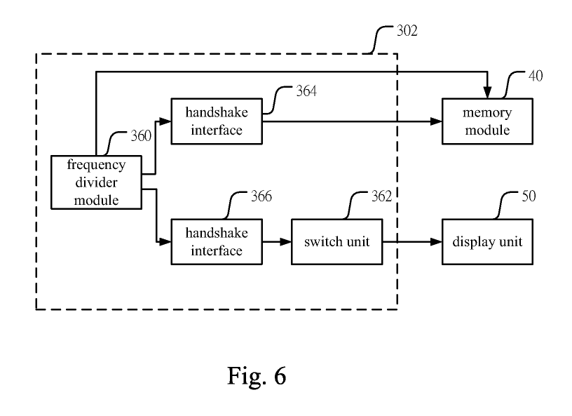

Refer to FIG. 6, compared with the 50 MHz clock frequency of the process control unit 30, the processing speed of the memory module 40 and the display unit 50 is far more slower. Thus once the process control unit 30 needs to control the memory module 40 as well as the display unit 50, its speed needs to match the execution speed of the memory module 40 as well as the display unit 50. Therefore, the peripheral control module 302 includes a frequency divider module 360, and a switch unit 362. The frequency divider module 360 receives ECG data and HRV parameter, reduces frequency of the ECG data as well as the HRV parameter and then saves them into the memory module 40. For example, by the frequency divider module 360, the system frequency such as 50 MHz of the process control unit 30 is reduced into 8.3 MHz so that it can be provided to the peripheral control module 302 for control of peripheral devices. Moreover, besides the computer 60 to show the stored ECG data and HRV parameter sent through the transmission interface 62, the medical device according to the present invention can also use the display unit 50 to show the data. Thus by means of the switch unit 362, the ECG and HRV data are switched to be shown by the display unit 50.

In addition, a handshake interface 364 is disposed between the frequency divider module 360 and the memory module 40 while another handshake interface 366 is between the frequency divider module 360 and the switch unit 362. The handshake interface 364 transmits ECG data to the memory module 40 by the handshake procedure while the handshake interface 366 is for transmitting ECG data and the HRV parameter to the switch unit 362 in the handshake way so that the data can be displayed by the display unit 50.

In summary, the present invention provides a medical device with real-time physiological signal analysis function. By means of the process control unit, the ECG data is processed to generate a HRV parameter. Then the ECG data and the HRV parameter are shown by a display unit. Thus changes of the HRV in the time domain as well as the frequency domain are analyzed and shown in time so that doctors can make diagnosis according to these data. Moreover, the process control unit is coupled to the memory module so as to save the ECG data and the HRV parameter. After long-term data collection of the ECG and HRV, doctors can make diagnosis by means of these data.

Additional advantages and modifications will readily occur to those skilled in the art. Therefore, the invention in its broader aspects is not limited to the specific details, and representative devices shown and described herein. Accordingly, various modifications may be made without departing from the spirit or scope of the general inventive concept as defined by the appended claims and their equivalents.

What is claimed is:

1. A medical device with real-time physiological signal analysis function comprising:
a detection circuit that detects a human heart to generate an electrocardiographic(ECG) signal;
a conversion circuit that receives the ECG signal and converts the ECG signal into an ECG data;
a process control unit for receiving and processing the ECG data so as to generate a HRV(Heart Rate Variability) parameter;
a memory module for saving the ECG data and the HRV parameter; and
a display unit that receives and displays the ECG data and the HRV parameter.

2. The device as claimed in claim 1, wherein the device further comprising:
a computer receiving the ECG data and the HRV parameter for display and storage of the ECG data and the HRV parameter.

3. The device as claimed in claim 2, wherein the device further comprising:
a transmission interface coupled to the computer and the process control unit for transmission of the ECG data and the HRV parameter.

4. The device as claimed in claim 3, wherein the transmission interface is a universal serial bus (USB) interface, a Peripheral Component Interconnect (PCI) card, a 1394 interface, a local area network (LAN) interface (IEEE802.3), an infrared (IrDA) interface, or a Bluetooth interface.

5. The device as claimed in claim 1, wherein the memory module comprising: a first memory unit and a second memory unit respectively for saving the ECG data and for saving the HRV parameter.

6. The device as claimed in claim 1, wherein the process control unit comprising:
an analysis process module that receives and processes the ECG data to generate the HRV parameter; and
a peripheral control module that receives the ECG data and the HRV parameter, and sends them to the memory module and the display unit.

7. The device as claimed in claim 6, wherein the process control unit further comprising: a keyboard module for control of the analysis process module and the peripheral control module to receive the ECG data and the HRV parameter.

8. The device as claimed in claim 7, wherein the process control unit further comprising:
a computation module that receives and processes the ECG data to generate a R-R interval;
a re-sampling unit that receives and samples the R-R interval to generate a equal sampled signal;
a Fourier Transform module that receives and turns the sampled signal to generate a spectrum signal; and
a square root calculation module that receives and processes the spectrum signal so as to generate the HRV parameter.

9. The device as claimed in claim 8, wherein the process control unit further comprising:
a handshake interface that receives and sends the ECG data to the computation module by a handshake procedure.

10. The device as claimed in claim 8, wherein the computation module comprising:
a first processing unit that receives and differentiates the ECG data and then takes the absolute value to get a differential data;
a second processing unit that receives and calculates the moving average of the differential data to generate a moving average data; and
a retrieving unit that detects the moving average data for retrieving a plurality of R waves to calculate the R-R interval.

11. The device as claimed in claim 8, wherein the Fourier Transform module comprising:
a Fourier Transform unit that turns the sampled signal into the spectrum signal having a real number and an imaginary number;
a first storage unit for saving the real number; and
a second storage unit for saving the imaginary number.

12. The device as claimed in claim 8, wherein the square root calculation module comprising:
a conversion unit that turns the real number and the imaginary number of the spectrum signal into an unsigned number of the spectrum signal;
a first processing unit for adding square of the unsigned number to get a square sum data; and
a second processing unit that takes the square root of the square sum data to generate the HRV parameter.

13. The device as claimed in claim 7, wherein the peripheral control module comprising:
a frequency divider module that receives the ECG data and the HRV parameter, reduces frequency of the ECG data as well as the HRV parameter and then reduced ECG data as well as reduced HRV parameter are saved into the memory module.

14. The device as claimed in claim 13, wherein the peripheral control module further comprising:
a handshake interface that receives and transmits the reduced ECG data to the memory module by a handshake procedure.

15. The device as claimed in claim 13, wherein the peripheral control module further comprising:
a switch unit that receives and switches the ECG and the HRV parameter into the display unit.

16. The device as claimed in claim 15, wherein the peripheral control module further comprising:
a handshake interface that receives and transmits the reduced ECG data and the reduced HRV parameter to the switch unit by a handshake procedure.

17. The device as claimed in claim 1, wherein the process control unit is a Field Programmable Gate Array (FPGA).

18. The device as claimed in claim 1, wherein the memory module is a flash memory.
